(19) United States
(12) Patent Application Publication
(10) Pub. No.: US 2020/0405793 A1
(43) Pub. Date: Dec. 31, 2020

(54) USE OF MEVALONATE METABOLIC PATHWAY INHIBITOR AND ALPHAVIRUS IN PREPARING ANTI-TUMOR DRUG

(71) Applicant: GUANGZHOU VIROTECH PHARMACEUTICAL CO., LTD., Guangzhou, Guangdong (CN)

(72) Inventors: Guangmei YAN, Guangzhou, Guangdong (CN); Jiankai LIANG, Guangzhou, Guangdong (CN); Wenbo ZHU, Guangzhou, Guangdong (CN); Haipeng ZHANG, Guangzhou, Guangdong (CN); Yuan LIN, Guangzhou, Guangdong (CN); Jing CAI, Guangzhou, Guangdong (CN); Shoufang GONG, Guangzhou, Guangdong (CN)

(21) Appl. No.: 16/959,094

(22) PCT Filed: Dec. 28, 2018
(86) PCT No.: PCT/CN2018/125012

§ 371 Date: Sep. 2, 2020

(30) Foreign Application Priority Data
Dec. 29, 2017 (CN) 201711483000.1

(51) Int. Cl. A61K 35/768; A61P 35/00

(52) U.S. Cl. CPC A61K 35/768; A61K 45/06; A61P 35/00

(57) ABSTRACT

Disclosed is the use of a mevalonate metabolic pathway inhibitor and an alphavirus in preparing an anti-tumor drug. The mevalonate metabolic pathway inhibitor can be used in preparing an alphavirus anti-tumor synergist. Disclosed are a pharmaceutical composition containing the mevalonate metabolic pathway inhibitor and the alphavirus, a drug kit containing the mevalonate metabolic pathway inhibitor and the alphavirus, and the use of the mevalonate metabolic pathway inhibitor and the alphavirus in treating tumors, especially those that are insensitive to the alphavirus.

### TECHNICAL FIELD

The present disclosure belongs to the field of biomedicine, and relates to use of a combination of mevalonate metabolic pathway inhibitor and an alphavirus in preparing an anti-tumor drug.

### BACKGROUND

Oncolytic virus is a class of replicable viruses that selectively infect and kill tumor cells without damaging normal cells. Oncolytic virus therapy is an innovative tumor targeted therapy strategy, which utilizes natural or genetically engineered viruses to selectively infect tumor cells and replicate in tumor cells to achieve targeted lysis and killing of tumor cells, but without damaging normal cells.

Alphavirus M1 belongs to the genus Alphavirus. M1 virus can selectively cause tumor cell death without affecting normal cell survival, and has a very good application prospect in anti-tumor aspect. However, different tumors have different sensitivities to M1 virus. For some tumors, when M1 virus is used alone, the oncolytic effect is not satisfactory. For example, as disclosed in the Chinese invention patent application 201410425510.3, when M1 is used as an antitumor drug, the effect on colorectal cancer, liver cancer, bladder cancer and breast cancer is less obvious than that on pancreatic cancer, nasopharyngeal carcinoma, prostate cancer and melanoma; on glioma, cervical cancer and lung cancer the effect is more inferior; and on gastric cancer, the effect is the least significant.

Screening for compounds that increase the therapeutic efficacy of oncolytic virus tumors would be expected to increase the antitumor spectrum and intensity of oncolytic virus. In the patent 201510990705.7 previously applied by the inventor, chrysophanol and derivatives thereof are used as anti-tumor synergists of oncolytic viruses, and the combination of the two can reduce the survival rate of tumor cells to 39.6%. However, there exists much room for improvement in its anti-cancer strength, and in addition, the action mechanism of the combined application is not clear.

The mevalonate metabolic pathway is one branch of the lipid anabolic system. Its upstream pathway is initiated with acetoacetyl-CoA, which under the action of HMG-CoA synthase (HMGCS1) produces 3-hydroxy-3-methylglutaryl-CoA (HMG-CoA), which is reduced by the rate-limiting enzyme HMG-CoA reductase to mevalonic acid, which under a series of enzymes produces farnesyl pyrophosphate (FPP). Its downstream pathway comprises three major metabolic pathways, including a cholesterol synthesis pathway, a protein farnesyl modification pathway involved in farnesylation modification of membrane proteins, and a protein geranylgeranylation modification pathway. A schematic of the mevalonate metabolic pathway can be seen in FIG. 9.

HMG-CoA reductase inhibitors are currently widely used clinically as lipid lowering agents, which not only reduce plasma cholesterol levels, but also prevent atherosclerosis.

Farnesyltransferase is a key enzyme for post-translational modification of Ras protein in cell signal transduction system. After translation of the Ras protein, farnesyl on the intermediate farnesyl pyrophosphate (FPP) in the cholesterol synthesis pathway is transferred to the CAAX tetrapeptide structure of the Ras protein under the catalysis of farnesyltransferase. The farnesyltransferase inhibitor can effectively inhibit farnesyl modification of Ras protein, thereby inhibiting the growth of tumors that dominate due to Ras gene activation.

Geranylgeranyltransferase is a key enzyme for post-translational modification of membrane proteins. After the protein is translated, the geranylgeranyl on the intermediate geranylgeranyl pyrophosphate (GGPP) in the mevalonate pathway is transferred to the CAAX tetrapeptide structure of the protein under the catalysis of geranylgeranyltransferase I/II, wherein the Rab geranylgeranyltransferase subunit beta specifically catalyzes the geranylgeranylation of the RAB protein.

It has been reported in the literature that mevalonate metabolic pathway plays an important role in promoting replication of various viruses. For example, upstream HMG-CoA reductase inhibitors such as statins can inhibit the replication of various viruses, including HCMV^{[1]}, HIV^{[2]}, and WNV^{[3]}. Downstream farnesyltransferase inhibitors can inhibit farnesylation modification of RAS, thereby inhibiting the replication of many viruses or their oncolytic effects, such as HSV-1^{[4]}.

### SUMMARY

It is an object of the present disclosure to provide use of a mevalonate metabolic pathway inhibitor in preparing an oncolytic virus alphavirus anti-tumor synergist.

It is another object of the present disclosure to provide an anti-tumor pharmaceutical composition which enables alphavirus to exert a better anti-tumor effect.

It is another object of the present disclosure to provide an alphavirus synergistic drug which is safe and effective against alphavirus insensitive tumors.

The present disclosure achieves the above objects through the following technical scheme:

The inventors have studied and screened that the mevalonate metabolic pathway inhibitor unexpectedly can enhance the oncolytic effect of alphavirus.

In the study of the inventor, the expression of 3-hydroxy-3-methylglutaryl coenzyme A reductase (HMGCR) gene or farnesyltransferase gene can be inhibited by a mevalonate metabolic pathway interference fragment (siRNA) to reduce the expressions of the corresponding proteins. The results showed that interfering mevalonate metabolic pathway alone and without interference would not cause cell morphological changes, and M1 virus applied alone would not cause cell morphological changes, only interfering mevalonate metabolism pathway combined with M1 virus group can cause significant cell morphological changes.

The inventors speculated that the oncolytic effect of alphavirus can be significantly enhanced by inhibiting the mevalonate metabolic pathway. Aiming at the presumption, the inventor adopted the compounds Tipifarnib, FTI277, fluvastatin and atorvastatin which inhibit mevalonate metabolic pathway activity to act on tumor cells in cooperation with the alphavirus, in particular M1 virus, and the experimental results showed that Tipifarnib, FTI277, fluvastatin and atorvastatin all can promote replication of the alphavirus Ml, thereby promoting cell death.

The mevalonate metabolic pathway inhibitor is a substance that inhibits the formation or activity of a metabolic initiator, or intermediate or end product in the mevalonate metabolic pathway, or a substance that degrades the metabolic initiator, or intermediate or end product in the mevalonate metabolic pathway, or a genetic tool that reduces the level of the metabolic initiator, or intermediate or end product in the mevalonate metabolic pathway.

The mevalonate metabolic pathway is one branch of the lipid anabolic system. Its upstream pathway is initiated with acetoacetyl-CoA, which under the action of HMG-CoA synthase (HMGCS1) produces 3-hydroxy-3-methylglutaryl-CoA (HMG-CoA), which is reduced by the rate-limiting enzyme HMG-CoA reductase to mevalonic acid, which under a series of enzymes produces farnesyl pyrophosphate (FPP). The downstream pathway of farnesyl pyrophosphate is divided into three major metabolic pathways, including cholesterol synthesis under the action of epoxidase, farnesylation modification of membrane proteins under the action of farnesyltransferase and geranylgeranylation modification under the action of geranylgeranyltransferase, to help proteins function normally.

Further, mevalonate metabolic pathway inhibitor in the present disclosure includes an upstream pathway inhibitor and/or downstream pathway inhibitor.

Wherein, the upstream pathway is a pathway initiated from acetoacetyl-CoA up to farnesyl pyrophosphate.

Specifically, acetoacetyl CoA and an acetyl CoA molecule are condensed under the catalysis of HMG-CoA synthase into HMG-CoA, which is reduced through the catalysis of HMG-CoA reductase to mevalonic acid; which is catalyzed by the mevalonate kinase and phosphomevalonate kinase to 3-phosphate-5-pyrophosphate mevalonate, which is then converted through mevalonate diphosphate decarboxylase to isopentenyl pyrophosphate or its isomer dimethylallyl pyrophosphate. Isopentenyl pyrophosphate and dimethylallyl pyrophosphate form geranyl pyrophosphate under the action of prenyltransferase, and further generate farnesyl pyrophosphate under the action of prenyltransferase.

Such upstream pathway inhibitors include substances that inhibits the activity or formation of both the metabolic initiators and products (intermediates, end products), e.g. any one or more of acetoacetyl-CoA, acetyl-CoA, 3-hydroxy-3-methylglutaryl-CoA, mevalonate, phosphomevalonate, pyrophosphomevalonate, isopentenyl pyrophosphate, dimethylacryldiphosphate (DMAPP), geranyl pyrophosphate (GPP) and farnesyl pyrophosphate (FPP) in the upstream pathway; and substances that inhibits the activity or formation of enzymes, e.g. any one or more of HMG-CoA synthase (HMGCS1), HMG-CoA reductase (HMGCR), mevalonate kinase (MVK), phosphomevalonate kinase (PMVK), mevalonate diphosphate decarboxylase (MVD), and farnesyl diphosphate synthase (FDPS) in the upstream metabolic pathway. (Of course, substances that degrade or knock down the above-mentioned targets are also inhibitors as described above.)

The downstream pathway inhibitor is a protein farnesyl modification pathway inhibitor and/or a geranylgeranylation modification pathway inhibitor. Further, the geranylgeranylation modification pathway inhibitor is a type II protein geranylgeranylation modification pathway inhibitor.

Still further, the protein farnesyl modification inhibitor is a farnesyltransferase inhibitor.

Still further, the type II protein geranylgeranylation modification pathway inhibitor is a geranylgeranyl pyrophosphate inhibitor; or preferably, the type II protein geranylgeranylation modification pathway inhibitor is a geranylgeranyl diphosphate synthase 1 (GGPS1) inhibitor and/or a geranylgeranyltransferase subunit beta (RABGGTB) inhibitor.

The mevalonate metabolic pathway inhibitor can be a substance (such as a compound, an amino acid sequence or a nucleotide sequence) which inhibits any link in the mevalonate metabolic pathway, wherein the link can be a metabolic initiator, an intermediate product or a final product, or can be an enzyme in the mevalonate metabolic pathway; or a substance that degradate products (including intermediates and end products), e.g. in particular, enzymes in the mevalonate metabolic pathway; or a tool (substance) capable of knocking out or affecting the amount of expression or activity of a protein in the mevalonate metabolic pathway. Those skilled in the art will be able to modify, replace and/or alter the inhibitory compound, sequence or genetic tool. If the substance obtained in the above manner has the effect of inhibiting the mevalonate metabolic pathway, then the substance belongs to the mevalonate metabolic pathway inhibitor described in the present disclosure, and belongs to the homogeneous replacement of the above substance, compound and tool in the present disclosure.

For the first time, the present disclosure discovers that the mevalonate metabolic pathway inhibitor can be used as an anti-tumor synergist/resistance reversal agent of the alphavirus.

The resistance reversal agent means that when some alphaviruses are used as antitumor drugs for treating tumors, some tumors are less sensitive to the alphaviruses, or the tumors are resistant to the alphaviruses, and in this case, an alphavirus combined with a mevalonate metabolic pathway inhibitor (as a drug resistance reversal agent) can be used to reverse the resistance of the tumors to the alphaviruses.

The present disclosure provides use of the mevalonate metabolic pathway inhibitor in preparing an alphavirus anti-tumor synergist.

As a preferred embodiment, the mevalonate metabolic pathway inhibitor is an inhibitor of an enzyme in the mevalonate metabolic pathway.

In a preferred embodiment of the present disclosure, the substance for use combined with the alphavirus as the anti-tumor synergist of the alphavirus is selected from at least one of an HMG-CoA reductase inhibitor, farnesyltransferase inhibitor and geranylgeranyltransferase inhibitor.

The geranylgeranyltransferase inhibitor is selected from a geranylgeranyltransferase subunit beta inhibitor.

The present disclosure provides use of one or more of HMG-CoA reductase inhibitor, farnesyltransferase inhibitor and geranylgeranyltransferase inhibitor in preparing an alphavirus anti-tumor synergist.

Wherein, the HMG-CoA reductase inhibitor, farnesyltransferase inhibitor, and geranylgeranyltransferase inhibitor are substances that inhibit activities of HMG-CoA reductase, farnesyltransferase, and geranylgeranyltransferase, or substances that degrade HMG-CoA reductase, farnesyltransferase, geranylgeranyltransferase, or genetic tools that reduce levels of HMG-CoA reductase and/or farnesyltransferase, geranylgeranyltransferase;

As an alternative embodiment, the substance that inhibits activity of HMG-CoA reductase is selected from a statin compound. It is well known in the art that the statin compound is an inhibitor of HMG-CoA reductase and is currently widely used in clinical lipid-lowering drugs, which not only can reduce plasma cholesterol levels but also prevent atherosclerosis. For the first time, the present disclosure discovers that the statin compound serving as an HMG-CoA reductase inhibitor can enhance the anti-tumor effect of the alphavirus.

As an illustrative example, the statin compound is selected from at least one of pravastatin, fluvastatin, lovastatin, simvastatin, atorvastatin, cerivastatin, rosuvastatin, and pitavastatin calcium, or a derivative thereof having an HMG-CoA reductase inhibitory effect, or a pharmaceutically acceptable salt, solvate, tautomer, or isomer thereof;

As a preferred embodiment of the present disclosure, the statin compound is selected from at least one of fluvastatin and atorvastatin or a derivative thereof having an HMG-CoA reductase inhibitory effect, or a pharmaceutically acceptable salt, solvate, tautomer, or isomer thereof;

As an embodiment of the present disclosure, the fluvastatin has a structural formula as shown in Formula I:

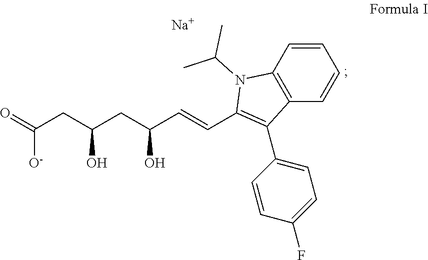 [SMILES: CC(C)n1c(/C=C/[C@@H](O)C[C@@H](O)CC(=O)[O-])c(-c2ccc(F)cc2)c2ccccc21 ; [Na+]]

as an embodiment of the present disclosure, the atorvastatin has a structural formula as shown in Formula II:

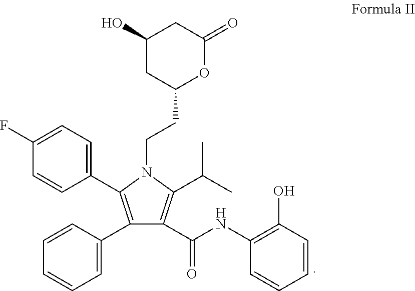

Wherein, the farnesyltransferase inhibitor is a known anti-hyperplasia agent. To date, there are a wide variety of known anti-hyperplasia agents, e.g. 5-fluorouracil, histone deacetylase (HDAC) inhibitor, cisplatin, vinblastine, estrogen receptor binding agent and the like. When attempting to use known anti-hyperplasia agents combined with alphaviruses, the results tend to be unpredictable. For example, the inventor have found that other anti-hyperplasia agents such as acetylase (HDAC) inhibitors do not produce a synergistic effect with alphaviruses. However, it is unexpected for farnesyltransferase inhibitors to produce synergistic antitumor effects with alphaviruses.

In of the present disclosure, the farnesyltransferase active substance employed is selected from one or more of a quinolinone, such as R115777; or benzodiazepine, such as BMS214662; or an aryl pyrrole, such as LB42908 and the like. These are non-competitive inhibitors of farnesyltransferase in the prior art.

Alternatively, the farnesyltransferase active substance is selected from, but not limited to, at least one of L-70472, J-104135, A-166120, Manumycin, and Chaetomium sp. acid, and other competitive inhibitors of farnesyltransferase, or a derivative thereof having a farnesyltransferase inhibitory effect, or a pharmaceutically acceptable salt, solvate, tautomer, or isomer.

As a preferred embodiment, the farnesyltransferase inhibitor in the present disclosure is selected from Tipifarnib and/or FTI277, or a pharmaceutically acceptable salt, solvate, tautomer, or isomer thereof.

As an embodiment of the present disclosure, the Tipifarnib has a structural formula as shown in Formula III:

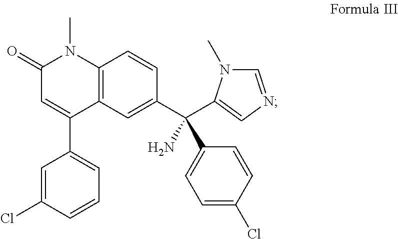 [SMILES: Cn1cncc1[C@@](N)(c1ccc(Cl)cc1)c1ccc2c(c1)c(-c1cccc(Cl)c1)cc(=O)n2C]

as an embodiment of the present disclosure, FTI277 has a structural formula as shown in Formula IV:

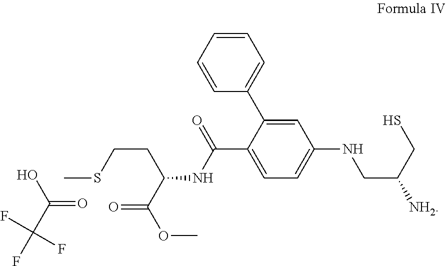

In some embodiments of the present disclosure, the mevalonate metabolic pathway inhibitor further includes a tool for inhibiting expression of the gene in the mevalonate metabolism pathway; preferably a tool for inhibiting expression of the enzyme gene in the mevalonate metabolism pathway; more preferably a tool for inhibiting expression of the gene of the HMG-CoA reductase, farnesyltransferase, or geranylgeranyltransferase, includeing, but not limited to, gene interference, and gene editing, gene silencing, or gene knockout.

Wherein, the geranylgeranyltransferase is a Rab geranylgeranyltransferase subunit beta. Correspondingly, the gene expression inhibition tool for geranylgeranyltransferase is a Rab geranylgeranyltransferase subunit beta gene expression inhibition tool.

As an alternative embodiment, the tools for inhibiting the expression of the enzyme gene in the mevalonate metabolism pathway, such as the tools for inhibiting the expression of HMG-CoA reductase, farnesyltransferase, or geranylgeranyltransferase genes, are selected from DNA, RNA, PNA or DNA-RNA-hybrid. They may be single-stranded or double-stranded.

These inhibitors can include small inhibitory nucleic acid molecules, such as short interfering RNA (siRNA), double-stranded RNA (dsRNA), microRNA (miRNA), ribozymes, and small hairpin RNA (shRNA), all capable of reducing or eliminating the expressions of proteins in the mevalonate metabolic pathway, especially the expressions of enzymes, more particularly the expression of HMG-CoA reductase, farnesyltransferase, or geranylgeranyltransferase.

These small inhibitory nucleic acid molecules may include first and second strands that hybridize to each other to form one or more double-stranded regions, each strand being about 18-28 nucleotides in length, about 18-23 nucleotides in length, or 18, 19, 20, 21, 22 nucleotides in length. Alternatively, a single strand may comprise regions capable of hybridizing to each other to form a double strand, such as in a shRNA molecule.

These small inhibitory nucleic acid molecules may include modified nucleotides while maintaining this ability to attenuate or eliminate the expressions of proteins, particularly enzymes, in the mevalonate metabolic pathway. Modified nucleotides can be used to improve in vitro or in vivo properties, such as stability, activity, and/or bioavailability. These modified nucleotides may contain a deoxynucleotide, 2′-methyl nucleotide, 2′-deoxy-2′-fluoronucleotide, 4′-trinucleotide, locked nucleic acid (LNA) nucleoside and/or 2′-O-methoxyethyl nucleotide, etc. Small inhibitory nucleic acid molecules, such as short interfering RNAs (siRNAs), may also contain a 5′-and/or 3′-cap structure to prevent degradation by an exonuclease.

In another preferred embodiment of the present disclosure, the mevalonate metabolic pathway inhibitor is an interfering RNA fragment of the mevalonate metabolic pathway; as an exemplary embodiment, it has the following sequence:

Gene for interfering HMG-CoA reductase

SEQ ID No: 1: AACCCAAUGCCCAUGUUCCdTdT

Gene for interfering farnesyltransferase

SEQ ID No: 2: ACGACTCGGTGGAAACAGT
SEQ ID No: 3: CGAGTTCTTTCACCTACTA

Interfering Rab geranylgeranyltransferase subunit beta

SEQ ID No: 4 SASI-HS01-00112524 (purchased from Sigma)

In some embodiments, a double-stranded nucleic acid consisting of a small inhibitory nucleic acid molecule comprises contain blunt or overhanging nucleotides at both ends. Other nucleotides may include nucleotides that result in dislocations, bumps, loops, or wobble base pairs. Small inhibitory nucleic acid molecules can be formulated for administration, e.g., by liposome encapsulation, or incorporation into other carriers (e.g., biodegradable polymer hydrogels, or cyclodextrins).

In other embodiments of the present disclosure, the inhibitor further comprises one or more of an antibody, an antibody functional fragment, a peptide, and peptoids. Preferred are one or more of antibodies, antibody functional fragments, peptides, and peptidomimetics that inhibit HMG-CoA reductase, farnesyltransferase or geranylgeranyltransferase.

Wherein the antibody can be a monoclonal antibody, a polyclonal antibody, a multivalent antibody, a multispecific antibody (e.g. bispecific antibodies). The antibody can be a chimeric antibody, a humanized antibody, a CDR-grafted antibody, or a human antibody. Antibody fragments can be, for example, Fab, Fab′, F(ab′) 2, Fv, Fd, single chain Fv (scFv), FV (sdFv) containing a disulfide-bond, or VL, VH domains. The antibody may be in a conjugated form, for example, bound to a tag, a detectable label, or a cytotoxic agent. The antibody may be a homotype IgG (e.g., IgG1, IgG2, IgG3, and IgG4), IgA, IgM, IgE or IgD.

Wherein, as of the present disclosure, the peptide inhibitor inhibiting farnesyltransferase is selected from a short peptide; more preferably, the short peptide is a tripeptide, tetrapeptide, pentapeptide, hexapeptide, heptapeptide or octapeptide; further, the peptide inhibitor is selected from one of CVFM or CIFM and the like. The peptidomimetic inhibitor for inhibiting farnesyltransferase, for example, based on the peptide inhibitors, can improve the defects of the peptide inhibitors through some techniques such as peptide bond conversion, group substitution and the like, improving the activity in cells, and improving the stability to peptidases.

The alphavirus can be M1 virus, the Getah virus, or a combination thereof.

The alphavirus (e.g., M1 virus, Getah virus, etc.) of the present disclosure may particularly refer to an existing alphavirus at present, but does not preclude viruses that have been naturally varied or mutated (natural, mandatory or selective mutation), genetic modified, sequence added, sequence added or deleted, or partial replaced. For example, an alphavirus having 99.8% or more, 99.5% or more, 99% or more, 98% or more, or even 97% or more homology. The alphaviruses described herein also include viruses that have undergone the above changes. Preferably, the above changes do not affect the alphavirus to function as described herein. The inhibitor for inhibiting the mevalonate metabolic pathway protein is a substance (such as a compound, or an amino acid sequence, a nucleotide sequence and the like) or a tool and the like capable of knocking down or influencing the gene expression or the protein amount or the protein activity of the mevalonate metabolic pathway. Modifications, substitutions, alterations and the like may be made by those skilled in the art to inhibit compounds or genetic tools thereof, but are intended to be protein inhibitors of the mevalonate metabolic pathway of the present disclosure, and are intended to be equivalent substitutions of the above substances, compounds or tools and the like, as long as they function to inhibit the mevalonate metabolic pathway as described above.

In some embodiments, the alphavirus is M1 virus deposited with Accession number CCTCC V201423 (deposited at the China Center for Type Culture Collection on Jul. 17, 2014). Genbank Accession No. EF011023, as a virus most likely derived from a same strain, records the sequence of M1. Getah virus is a virus having 97.8% homology to M1 virus (Wen et al. Virus Genes. 2007; 35(3):597-603). There is high identity between the two, and M1 virus is also classified as Getah-like virus by some literature. It is expected that there is more similar properties between the two.

A single alphavirus strain may also be administered. In other embodiments, multiple viral strains and/or types of alphaviruses may also be used.

The present disclosure also provides a pharmaceutical composition for treating tumors comprising a mevalonate metabolic pathway inhibitor and an alphavirus.

The present disclosure also provides a drug kit for treating tumors comprising a mevalonate metabolic pathway inhibitor or derivative thereof, or a combination thereof, and an alphavirus.

The difference between the drug kit and the composition is that in the drug kit, the mevalonate metabolic pathway inhibitor and the alphavirus preparation are packaged separately (e.g. a pill, or capsule, or tablet or ampoule contains the mevalonate metabolic pathway inhibitor; and an additional pill, or capsule, or tablet or ampoule contains the alphavirus). In some embodiments, the alphavirus, the mevalonate metabolic pathway inhibitor, and the combination of the alphavirus and the mevalonate metabolic pathway inhibitor may also contain one or more adjuvants. The adjuvant refers to a component which can assist the efficacy of the medicine in the pharmaceutical composition. The drug kit may also comprise an independently packaged mevalonate metabolic pathway inhibitor, as well as an independently packaged alphavirus. The mevalonate metabolic pathway inhibitor, as well as the alphavirus in the drug kit, may be administered simultaneously or sequentially in any order, e.g., the mevalonate metabolic pathway inhibitor is administered before or after the alphavirus, or both are administered simultaneously. In various embodiments, the patient may be a mammal. In some embodiments, the mammal may be a human.

As a preferred embodiment, the mevalonate metabolic pathway inhibitor is selected from compounds that inhibit activity of the mevalonate metabolic pathway, such as fluvastatin (Formula I), atorvastatin (Formula II), Tipifarnib (Formula III), FTI277 (Formula IV). Or the tool for inhibiting gene expression of the mevalonate metabolic pathway includes, but not limited to, tool means for gene interference, gene silencing, and gene editing or knockout.

The alphavirus is selected from M1 virus, Getah virus, or a combination thereof.

The ratio of the inhibitor (e.g., Tipifarnib, FTI277, fluvastatin or atorvastatin) to alphavirus in the composition or drug kit is optionally: 0.01-200 mg: 10³-10⁹ PFU; preferably 0.1-200 mg: 10⁴-10⁹ PFU; more preferably 0.1-100 mg: 10⁵-10⁹ PFU;

preferably, the inhibitor (for example, Tipifarnib, FTI277, fluvastatin or atorvastatin) is used in a range of 0.01 mg/kg to 200 mg/kg, while the alphavirus is used at a titer of MOI from 10³ to 10⁹ (PFU/kg); in some embodiments, the alphavirus is used at a titer of MOI of 10³-10⁴ or 10⁴-10⁵ or 10⁵-10⁶ or 10⁶-10⁷ or 10⁷-10⁸ or 10⁸-10⁹ PFU/kg. Preferably, the inhibitor (for example, Tipifarnib, FTI277, fluvastatin or atorvastatin) is used in a range of 0.1 mg/kg to 200 mg/kg, while the alphavirus is used at a titer of MOI from 10⁴ to 10⁹ (PFU/kg); more preferably, the inhibitor (for example, Tipifarnib, FTI277, fluvastatin or atorvastatin) is used in a range of 0.1 mg/kg to 200 mg/kg, while the alphavirus is used at a titer of MOI from 10⁵ to 10⁹ (PFU/kg); more preferably, the inhibitor (for example, Tipifarnib, FTI277, fluvastatin or atorvastatin) is used in a range of 0.1 mg/kg to 100 mg/kg, while the alphavirus is used at a titer of MOI from 10⁵ to 10⁹ (PFU/kg).

In, the tumor is a solid tumor or a hematologic tumor. In , the solid tumor is liver cancer, colorectal cancer, bladder cancer, breast cancer, cervical cancer, prostate cancer, glioma, melanoma, pancreatic cancer, nasopharyngeal cancer, lung cancer, or gastric cancer. In a preferred embodiment, the tumor is an alphavirus insensitive tumor. In a more preferred embodiment, the tumor is an M1 virus insensitive tumor.

As an alternative embodiment, the inhibitors provided herein (e.g., Tipifarnib, FTI277, fluvastatin or atorvastatin, or combinations thereof) can be injections, tablets, capsules, patches, and the like. As a preferred embodiment, the synergistic agent of the present disclosure is an injection; preferably, intravenous injection may be used.

The present disclosure discloses a mevalonate metabolic pathway inhibitor, in particular to Tipifarnib, FTI277, fluvastatin or atorvastatin, which can increase the replication and anti-tumor effect of alphavirus and improve the therapeutic effectiveness of alphavirus as an anti-tumor medicament. Cytological experiments showed that M1 virus combined with Tipifarnib and FTI277 could significantly induce the morphological changes of tumor cells, thus significantly enhance the inhibition of tumor cells. Biomolecular experiments show that M1 virus combined with fluvastatin or atorvastatin respectively, or interfering HMGCR, FNTB or RABGGTB can significantly increase the protein expression of M1 virus, thereby enhancing the inhibitory effect of M1 virus on tumor cells.

The HMGCR gene targeting mevalonic acid metabolic pathway by siRNA was used, and colorectal cancer cells HCT-116 cells, pancreatic cancer cells Capan-1 cells and SW1990 cells were adherently grown under the treatment of disordered interference fragments; the interfering HMGCR did not affect the morphology of tumor cells; in the case of infection with M1 virus alone, a small amount of cell death was observed under microscope; however, a large number of cell death was observed in cells of the HMGCR interference group 48 hours after M1 infection. The survival rate of HCT-116 cells was detected by MTT assay. In HCT-116 cells, infection with M1 virus alone caused about 20% cell death; however, infection with M1 virus can cause more than 70% cell death following interfering HMG-CoA reductase. The tumor killing effect of M1 virus is obviously enhanced.

We combined Tipifarnib or FTI277 and M1 virus to treat human intestinal cancer cell HCT-116 strain. We have surprisingly found that Tipifarnib or FTI277 combined with M1 virus can significantly increase the morphological lesions of tumor cells and significantly reduce the survival rate of tumor cells. For example, in an embodiment of the present disclosure, when intestinal cancer cells were treated with M1 virus (MOI=1) alone, the tumor cell survival was 97.0%, and when treated with 50 nM Tipifarnib combined with M1 virus of the same MOI, the tumor cell survival was greatly reduced to 38%. Compared with the anti-tumor effect of M1 virus administrated alone, Tipifarnib combined with M1 significantly improved the oncolytic effect.

The present disclosure has found that treatment of tumor cells with Tipifarnib or FTI277 combined with alphavirus has a significantly better killing effect on tumor cells than treatment with the same concentration of Tipifarnib or FTI277 alone, e.g., the tumor cell survival was still as high as 80% when tumor cells were also treated, e.g., with 50 nM Tipifarnib, and the tumor cell survival wais greatly reduced to 38% when 50 nM Tipifarnib was used combined with M1 virus. Therefore, when the Tipifarnib is combined with M1, the greatly improved oncolytic effect benefits from the synergistic mechanism between the Tipifarnib and M1 virus, rather than the role played through the anti-tumor mechanism of the Tipifarnib.

### BRIEF DESCRIPTION OF THE DRAWINGS

FIG. 1 siRNA targeting 3-hydroxy-3-methylglutaryl-CoA reductase HMGCR and M1 virus significantly increase the morphological lesions of human intestinal cancer and pancreatic cancer cell strains;

FIG. 2 Treatment of siRNA targeting 3-hydroxy-3-methylglutaryl-CoA reductase HMGCR combined with M1 virus significantly reduces the survival rate of the human intestinal cell carcinoma strain.

FIG. 3 The statin and M1 virus significantly increase morphological lesions of human intestinal cell carcinoma strains and promote replication of M1 virus; wherein A shows a phase difference diagram of the human intestinal cell carcinoma strain after fluvastatin and M1 treatment; and B shows fluvastatin and atorvastatin promote the expression of M1 virus protein.

FIG. 4 Treatment of siRNA targeting subunit FNTB of farnesyltransferase combined with M1 virus significantly increases morphological lesions in the human intestinal cell carcinoma strain and pancreatic cancer cell strain.

FIG. 5 The farnesyltransferase inhibitors Tipifarnib, FTI277 and M1 significantly increase morphological lesions in the human intestinal cell carcinoma strain and pancreatic cancer cell strain.

FIG. 6 Treatment of the farnesyltransferase inhibitor Tipifarnib combined with M1 virus significantly reduce the survival of the human intestinal cell carcinoma strain and pancreatic cancer cell strain.

FIG. 7 Interfering the upstream pathway and four branches of downstream pathway of the mevalonate pathway to screen that blocking of farnesylation and geranylgeranylation pathways in downstream branches promotes replication of M1 virus.

FIG. 8 The farnesyltransferase inhibitor Tipifarnib combined with M1 virus significantly inhibited the growth of transplanted tumors of the human intestinal cell carcinoma strain and pancreatic cell carcinoma strain; wherein A shows a growth curve of the transplanted tumor of the human intestinal cell carcinoma strain; and B shows a growth curve of the transplanted tumor of the human pancreatic cell carcinoma strain.

FIG. 9. Mevalonic acid metabolic pathway diagram

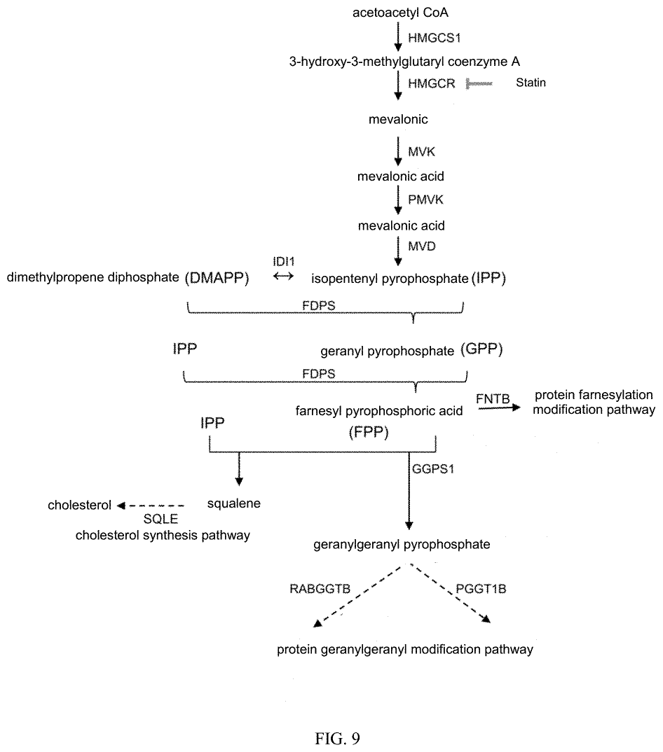

The full names corresponding to the English abbreviations for the enzymes referred to in FIG. 9 are as follows:

HMGCR: 3 -hydroxy-3-methylglutaryl-CoA reductase

HMGCS1: 3-hydroxy-3-methylglutaryl-CoA synthase 1 HM

MVK: mevalonate kinase

PMVK: phosphomevalonate kinase

MVD: mevalonate diphosphate decarboxylase

IDI1: isopentenyl-diphosphate delta isomerase 1

FDPS: farnesyl diphosphate synthase

GGPS1: geranylgeranyl diphosphate synthase 1

DHCR 7: 7-dehydrocholesterol reductase

RAGBBTB: Rab geranylgeranyltransferase subunit beta

PGGT1B: protein geranylgeranyltransferase type I subunit beta

FNTB: farnesyltransferase, CAAX box, beta

SQLE: squalene epoxidase

### DETAILED DESCRIPTION

The following embodiments further illustrate the present disclosure, but the embodiments of the present disclosure are not limited to the following examples. Any equivalent changes or modifications made in accordance with the principles or concepts of the present disclosure should be regarded as the scope of protection of the present disclosure.

Without being specifically indicated, the materials and experimental methods employed in the present disclosure are conventional materials and methods.

The term “selected from” in the specification is used in connection with a selected object and is to be understood as, for example: “x is selected from: A, B, C, . . . E” or “X is selected from one or more of A, B, C, . . . and E”, and the like, are understood to mean that X comprises one, or any combination of two, or any combination of more of A, B, C, . . . E. It is not excluded that X also includes some other class of substances.

In addition to the specific enzyme inhibitors mentioned above, the inhibitors of the present disclosure may be selected from specific enzyme inhibitors already known in the art, or substances found to have specific enzyme inhibition after subsequent studies. For example, with respect to farnesyltransferase inhibitors, the farnesyltransferase inhibitors of the present disclosure may also be selected from farnesyltransferase inhibitors known in the art, or substances found to have farnesyltransferase inhibitory effects upon subsequent studies. The same holds true for HMG-CoA reductase inhibitors, geranylgeranyltransferase or other specific enzyme inhibitors.

The enzymes involved in the various mevalonate metabolic pathways exemplified in the present disclosure are as follows, along with their known sequences (reported in NCBI, below are NCBI Gene IDs).

HMGCR: 3-hydroxy-3-methylglutaryl-CoA reductase ID:3156

HMGCS1: 3-hydroxy-3-methylglutaryl-CoA synthase 1 HM ID:3157

MVK: mevalonate kinase ID:4598

PMVK: phosphomevalonate kinase ID:10654

MVD: mevalonate diphosphate decarboxylase ID:4597

IDI1: isopentenyl-diphosphate delta isomerase 1 ID:3422

FDPS: farnesyl diphosphate synthase ID:2224

GGPS1: geranylgeranyl diphosphate synthase 1 ID:9453

DHCR7: 7-dehydrocholesterol reductase ID:1717

RAGBBTB: Rab geranylgeranyltransferase subunit beta ID:5876

PGGT1B: protein geranylgeranyltransferase type I subunit beta ID:5229

FNTB: farnesyltransferase, CAAX box, beta ID:2342

SQLE: squalene epoxidase ID:6713

Of course, the above sequences are not intended to be limiting. Since it is not excluded that are newly discovered and perform similar functions, or other analogs and the like, which may vary in amino acid sequence or nucleotide sequence, for example, proteins with a sequence identity of at least 80%, at least 85%, at least 90%, at least 91%, at least 92%, at least 93%, at least 94%, at least 95%, at least 96%, at least 97%, at least 98%, or at least 99%, or at least 99.5%, or at least 99.8%, they may be subsequently found to achieve similar functions, which belong to the above-mentioned analogs. Inhibitors designed for them are also within the scope of protection of the present disclosure.

### EXAMPLE 1 TREATMENT OF siRNA TARGETING 3-HYDROXY-3-METHYLGLUTARYL CoA REDUCTASE (HMGCR) COMBINED WITH M1 VIRUS SIGNIFICANTLY INCREASED MORPHOLOGICAL LESIONS IN HUMAN INTESTINAL CANCER AND PANCREATIC CANCER CELL STRAINS

### Materials

Human intestinal cell carcinoma strain HCT-116 (purchased from the Cell Bank of the Chinese Academy of Sciences), human pancreatic cancer cells Capan-1 (purchased from ATCC), SW 1990 (purchased from ATCC), M1 virus (Accession number CCTCC V201423), high glucose DMEM medium (purchased from Corning), and inverted phase contrast microscope.

### Method

The cells were inoculated into a 35 mm culture dish, cultured until the confluence degree of the cells reaches 60% and subjected to the following interference treatment: firstly, a Lipofectamine RNAiMAX solution was prepared with Opti-MEM by diluting according to 2 μL: 198 μL per culture dish and uniformly mixing; secondly, an siRNA solution was prepared with Opti-MEM by diluting according to 1.8 μL: 198 μL per culture dish, wherein the final concentration of siRNA was 25 nM, and mixing gently; finally, the diluted Lipofectamine RNAiMAX and siRNA were mixed, and stood for 15 minutes at room temperature; the mixed solution was added into a culture dish containing 1.5 mL of serum-free culture medium; and the medium was changed to a complete medium after 24 hours, and infected with M1 virus (1MOI). The changes in cell morphology were observed after 48 h under the inverted phase contrast microscope.

The sequence of the siRNA is as follows:

SEQ ID No: 1: AACCCAAUGCCCAUGUUCCdTdT

### Result

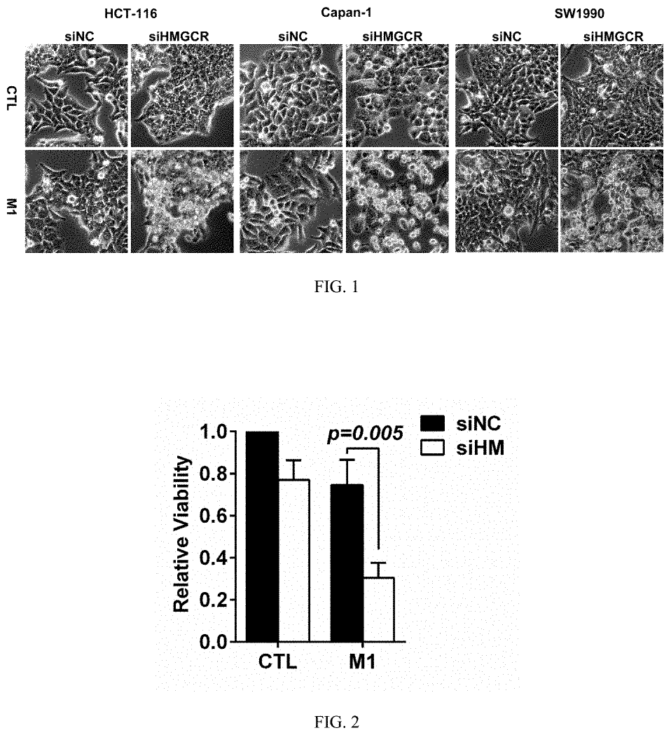

As shown in FIG. 1, colorectal cancer cells HCT-116, pancreatic cancer cells Capan-1 and SW 1990 cells grew well adherently under the treatment of disordered interfering fragments; the interfered HMGCR did not affect the morphology of tumor cells; in the case of infection with M1 virus alone, a small number of cells were observed under the microscope to be shrunk, rounded, and enhanced in refractive index; however, after 48 hours of M1 infection, cells in the HMGCR interference group obviously showed a large number of dead cells with enhanced refractive index, floating or adhering to the culture dish.

### EXAMPLE 2 TREATMENT OF siRNA TARGETING 3-HYDROXY-3-METHYLGLUTARYL-CoA REDUCTASE HMGCR COMBINED WITH M1 VIRUS SIGNIFICANTLY REDUCED THE SURVIVAL RATE OF HUMAN INTESTINAL CANCER CELL STRAINS

### Materials

Human intestinal cell carcinoma strain HCT-116 (purchased from the Cell Bank of the Chinese Academy of Sciences), M1 virus (Accession number CCTCC V201423), and high glucose DMEM medium (purchased from Corning).

### Method

a) Cell culture: human intestinal cell carcinoma strain HCT-116 was grown in a DMEM complete medium containing 10% FBS, 100 U/ml penicillin and 0.1 mg/ml streptomycin; all cell strains were cultured in a constant temperature closed incubator at 5% CO₂, 37° C. (95% RH) and observed by the inverted microscope. Cells were passaged once for approximately 2-3 days and the cells in logarithmic growth phase were used for formal experiments.

b) The cells were inoculated into a 24-well plate at 30,000 cells/well; the cells were infected with M1 virus (MOI=1) after 24 hours of interference treatment with siRNA targeting HMGCR; 72 hours after infection, the cell survival rate was detected by MTT assay, which comprises the following steps: MTT solution was added at 100 μL/well; after incubation for 3 hours at 37° C., the supernatant was aspirated off and DMSO solution was added at 1 mL/well; after shaking well, the plate was placed in a microplate reader and the absorbance was measured at 570 nm.

### Result

As shown in FIG. 2, infection with M1 virus alone caused about 20% cell death in HCT-116 cells; however, infection with M1 virus can cause more than 70% cell death following interfering the HMG-CoA reductase gene (HMGCR).

The sequence of the siRNA is as follows:

SEQ ID No: 1: AACCCAAUGCCCAUGUUCCdTdT

### EXAMPLE 3 THE STATIN AND M1 VIRUS SIGNIFICANTLY INCREASED MORPHOLOGICAL LESIONS OF HUMAN INTESTINAL CELL CARCINOMA STRAINS AND PROMOTED REPLICATION OF M1 VIRUS

### Materials

Human intestinal cell carcinoma strain HCT-116 (purchased from the Cell Bank of the Chinese Academy of Sciences), M1 virus (Accession number CCTCC V201423), high glucose DMEM medium (purchased from Corning), and inverted phase contrast microscope.

### Method

a) Cell culture: human intestinal cell carcinoma strain HCT-116 was grown in a DMEM complete medium containing 10% FBS, 100 U/ml penicillin and 0.1 mg/ml streptomycin; all cell strains were cultured in a constant temperature closed incubator at 5% CO₂, 37° C. (95% RH) and observed by the inverted microscope. Cells were passaged once for approximately 2-3 days and the cells in logarithmic growth phase were used for formal experiments.

b) Cell treatment and morphological observation: cells in logarithmic phase growth were selected, prepared as a cell suspension in DMEM complete medium (containing 10% fetal bovine serum, and 1% double antibody) and inoculated into a 24-well plate at a density of 4×10⁵/well. Cells were infected with M1 virus (MOI=1), treated with M1 virus (MOI=1) combined with fluvastatin or atorvastatin (1 and 10 μM), and cellular proteins were harvested after 24 hours and immunoblotted.

c) Cells were inoculated into a 24-well plate at 30,000 cells/well; after the cells were treated with fluvastatin 2 (μM), they were infected with M1 virus (MOI=1); and the changes of cell morphology were observed under the inverted phase contrast microscope after 48 hours.

### Result

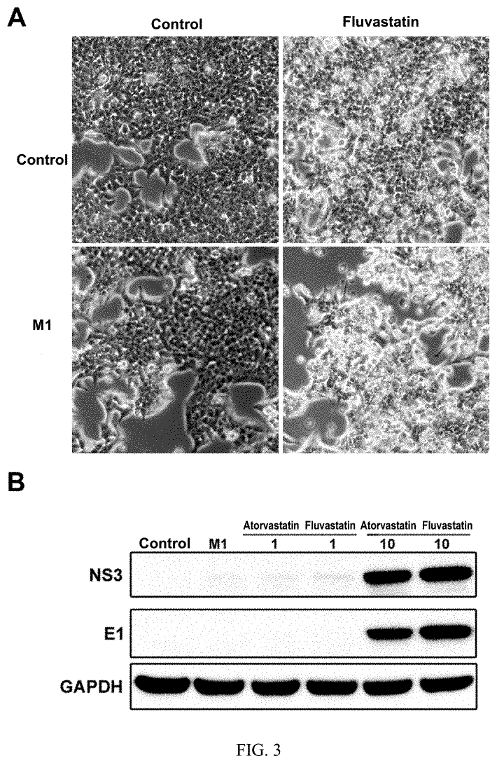

FIG. 3A shows that M1 alone had no significant effect on cell morphology, fluvastatin alone induced lesions in a small number of cells, and when the cells were treated with the two drugs at the same time, significant lesions in cells occur; as shown in FIG. 3B, M1 viral proteins were slightly up-regulated at a drug concentration of 1 μM; when the concentration of statins was increased to 10 μM, the expression of M1 viral proteins (structural protein El and non-structural protein NS3) were significantly up-regulated compared to the drug-free group. The result demonstrated that statins promote expression of M1 viral proteins.

### EXAMPLE 4 TREATMENT OF siRNA TARGETING SUBUNIT FNTB OF FARNESYLTRANSFERASE COMBINED WITH M1 VIRUS SIGNIFICANTLY INCREASED MORPHOLOGICAL LESIONS IN HUMAN INTESTINAL AND PANCREATIC CANCER CELL STRAINS

### Materials

Human intestinal cell carcinoma strain HCT-116 (purchased from the Cell Bank of the Chinese Academy of Sciences), human pancreatic cell carcinoma strain Capan-1 (purchased from ATCC), SW 1990 (purchased from ATCC), M1 virus (Accession number CCTCC V201423), high glucose DMEM medium (purchased from Corning), and inverted phase contrast microscope.

### Method

The cells were inoculated into a 35 mm culture dish, cultured until the confluence degree of the cells reaches 60% and subjected to the following interference treatment: firstly, a Lipofectamine RNAiMAX solution was prepared with Opti-MEM by diluting according to 2 μL: 198 μL per culture dish and uniformly mixing; secondly, an siRNA solution was prepared with Opti-MEM by diluting according to 1.8 μL: 198 μL per culture dish, wherein the final siRNA concentration was 10 or 2 nM, and mixing gently; finally, the diluted Lipofectamine RNAiMAX and siRNA were mixed, and stood for 15 minutes at room temperature; the mixed solution was added into a culture dish containing 1.5 mL of serum-free culture medium; and the medium was changed to a complete medium after 24 hours, and infected with M1 virus (1MOI). The changes in cell morphology were observed after 48 h under the inverted phase contrast microscope.

The sequence of the siRNA is as follows:

SEQ ID No: 2: ACGACTCGGTGGAAACAGT
SEQ ID No: 3: CGAGTTCTTTCACCTACTA

### Result

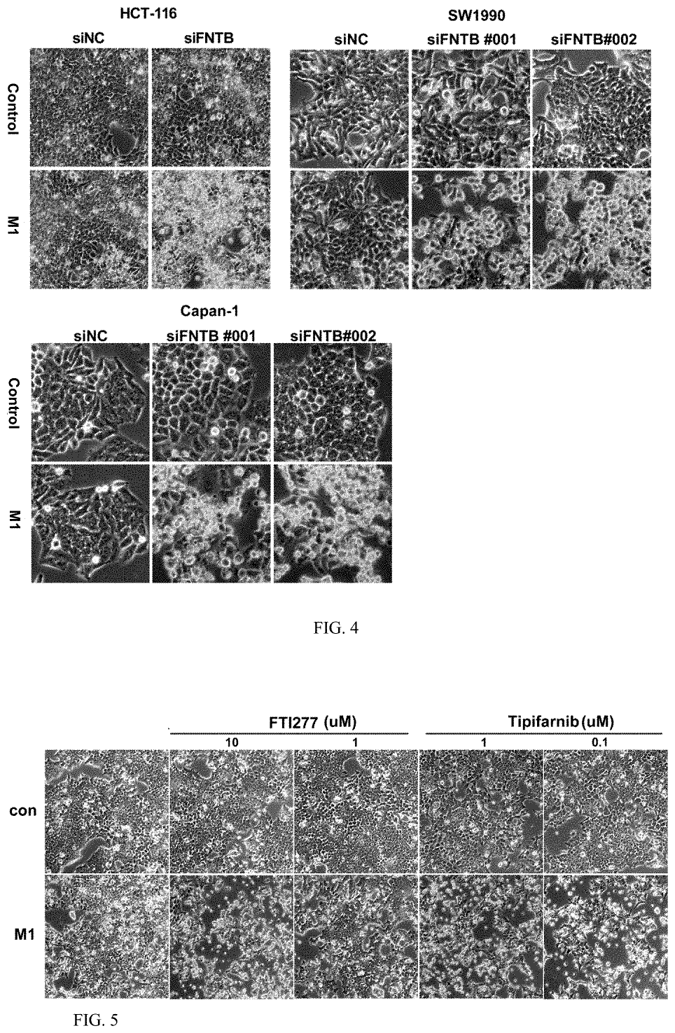

As shown in FIG. 4, the colorectal cancer cells HCT-116, pancreatic cancer cells Capan-1 and SW1990 cells grew well adherently under the treatment of disordered interfering fragments; the interfered FNTB did not affect the morphology of tumor cells; in the case of infection with M1 virus alone, a small number of cells were observed under the microscope to be shrunk, rounded, and enhanced in refractive index; however, after 48 hours of M1 infection, cells in the FNTB interference group obviously showed a large number of dead cells with enhanced refractive index, floating or adhering to the culture dish.

### EXAMPLE 5 THE FARNESYLTRANSFERASE INHIBITORS TIPIFARNIB, FTI277 and M1 VIRUS SIGNFICANTLY INCREASED MORPHOLOGICAL LESIONS OF HUMAN INTESTINAL CELL CARCINOMA STRAINS

### Materials

Human intestinal cell carcinoma strain HCT-116 (purchased from the Cell Bank of the Chinese Academy of Sciences), M1 virus (Accession number CCTCC V201423), high glucose DMEM medium (purchased from Corning), and inverted phase contrast microscope.

### Method

a) Cell culture: human intestinal cell carcinoma strain HCT-116 was grown in a DMEM complete medium containing 10% FBS, 100 U/ml penicillin and 0.1 mg/ml streptomycin; all cell strains were cultured in 5% CO2, 37° C. incubator (95% RH) and observed by the inverted microscope. Cells were passaged once for approximately 2-3 days and the cells in logarithmic growth phase were used for formal experiments.

b) Cell treatment and morphological observation: cells in logarithmic phase growth were selected, prepared as a cell suspension in DMEM complete medium (containing 10% fetal bovine serum, and 1% double antibody) and inoculated into a 24-well plate at a density of 2.5×10⁴/well. Cells were treated with Tipifarnib (1, 0.1 μM) alone, FTI277 (10, 1 μM) alone, M1 virus (MOI=1) infection, M1 virus (MOI=1) combined with Tipifarnib (1, 0.1 μM), M1 virus (MOI=1) combined with FTI277 (10, 1 μM), with no addition of M1 virus, FTI277 and Tipifarnib as a control, and the cell morphology changes were observed under the inverted phase contrast microscope after 48 hours.

### Result

As shown in FIG. 5, the morphology of the cells was observed under the phase contrast microscope, HCT-116 cells were monolayer adherently grown, and the cells were closely arranged with a consistent phenotype. After 48 h of treatment with FTI1277 or Tipifarnib (50 nM) and M1 virus (MOI=1), the morphology of the cells changed significantly. Compared with the control group cells, M1 solely treatment group and other solely treatment groups, the number of cells in the combined treatment group decreased significantly, the cell body contracted into globular shape, and the refractive index increased significantly, and death-like lesions are showed.

### EXAMPLE 6 TREATMENT OF THE FARNESYLTRANSFERASE INHIBITOR TIPIFARNIB COMBINED WITH M1 VIRUS SIGNIFICANTLY REDUCED THE SURVIVAL OF HUMAN INTESTINAL CANCER AND PANCREATIC CANCER CELL STRAINS

### Materials

Human intestinal cell carcinoma strain HCT-116 (purchased from the Cell Bank of the Chinese Academy of Sciences), human pancreatic carcinoma cell strain SW1990 (purchased from ATCC), human normal liver cell strain L-02 (purchased from the Cell Bank of the Chinese Academy of Sciences), M1 virus (accession number CCTCC V201423), high-glucose DMEM medium (purchased from Corning), and automatic enzyme-linked detection microplate reader.

### Method

a) Cells inoculation, and administration treatment: cells in logarithmic phase growth were selected, prepared as a cell suspension in DMEM complete medium (containing 10% fetal bovine serum, and 1% double-antibody) and inoculated into 96-well plates at a density of 4×10³/well. After 12 hours, the cells were seen completely adherent and divided into control group, Tipifarnib alone group, M1 infection group and Tipifarnib/M1 combination group. The dosages used were: M1 virus (MOI =1) infected cells: Tipifarnib (50 nM).

B) Reaction of MTT with intracellular succinate dehydrogenase: after 48 h of culture, 20 μl of MTT (5 mg/ml) was added to each well and the incubation was continued for 4 h, at which point microscopic examination revealed the formation of granular blue-violet formazan crystals in living cells.

c) Dissolution of formazan particles: the supernatant was carefully aspirated off, DMSO 100 μl/well was added to dissolve the crystals formed, shaken on a microshaker for 5 min, and the optical density (OD) of each well was measured on the enzyme-linked detector at a wavelength of 570 nm. Each group of experiments was repeated 3 times. Cell viability=OD of drug treated group/OD of control group×100%.

### Result

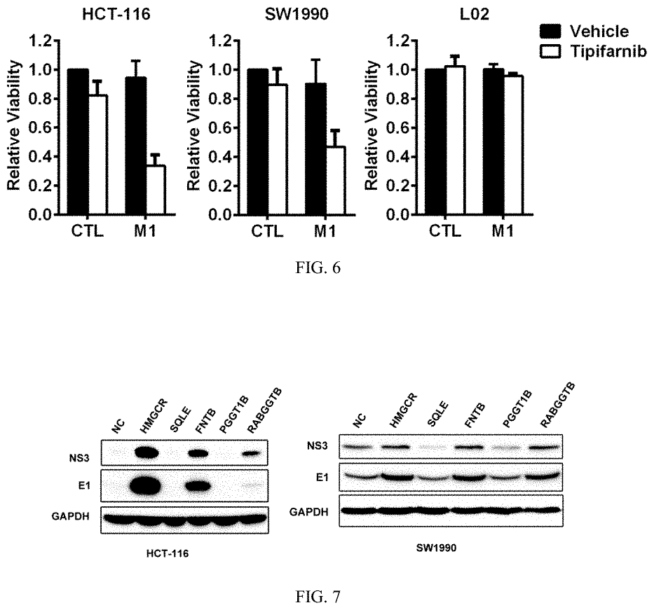

As shown in FIG. 6, M1 virus alone treatment had a small survival rate inhibition effect on tumor cell HCT-116 as the tumor cell survival rate reached 97.0%, the tumor cell survival rate of the 50 nM Tipifarnib treatment group still reached 80%, however, when the 50 nM Tipifarnib was used combined with M1 virus of the same MOI (Tipifarnib+M1), the tumor cell survival rate was greatly reduced to 38%; M1 virus alone treatment had a small survival rate inhibition effect on tumor cell SW190 as the tumor cell survival rate reached 90%, the tumor cell survival rate of the 50 nM Tipifarnib treatment group still reached 90%, however, when the 50 nM Tipifarnib was used combined with M1 virus of the same MOI (Tipifarnib+M1), the tumor cell survival rate was greatly reduced to about 40%; and the combined treatment had no obvious killing effect on L02 cells. It is thus demonstrated at the cellular level that the mevalonate metabolic pathway protein inhibitor can enhance the oncolytic effect of M1 virus without killing normal cells.

### EXAMPLE 7 INTEFERING THE UPSTREAM PATHWAY AND FOUR BRANCHES OF DOWNSTREAM OFO THE MEVALONATE PATHWAY AND SCREENINGN FARNESYLATION AND GERANYLGERANYLATION PATHWAYS IN DOWNSTREAM BRANCHES TO BE BLOCKED TO PROMOTE REPLICATION OF M1 VIRUS

### Materials

Human intestinal cell carcinoma strain HCT-116 (purchased from the Cell Bank of the Chinese Academy of Sciences), M1 virus (Accession number CCTCC V201423), high glucose DMEM medium (purchased from Corning), and inverted phase contrast microscope.

### Method

The cells were inoculated into a 35 mm culture dish, cultured until the confluence degree of the cells reaches 60% and subjected to the following interference treatment: firstly, a Lipofectamine RNAiMAX solution was prepared with Opti-MEM by diluting according to 2 μL: 198 μL per culture dish and uniformly mixing; secondly, an siRNA solution was prepared with Opti-MEM by diluting according to 1.8 μL: 198 μL per culture dish, wherein the final concentration of siRNA interfering HMGCR, SQLE, FNTB, PGGT 1B and RABGGTB (specifically see FIG. 9 and the description of the Figures) were 25, 25, 4, 20 and 20 nM respectively, and mixing gently; finally, the diluted Lipofectamine RNAiMAX and siRNA were mixed, and stood for 15 minutes at room temperature; the mixed solution was added into a culture dish containing 1.5 mL of serum-free culture medium; and the medium was changed to a complete medium after 24 hours, and infected with M1 virus (1MOI). Cell lysates were collected after 24 hours and immunoblotted.

The siRNAs are as follows:

Gene for interfering HMG-CoA reductase (HMGCR)

SEQ ID No: 1: AACCCAAUGCCCAUGUUCCdTdT

Interfering farnesyltransferase subunit FNTB

SEQ ID No: 2: ACGACTCGGTGGAAACAGT
SEQ ID No: 3: CGAGTTCTTTCACCTACTA

Interfering Rab geranylgeranyltransferase subunit beta

SEQ ID No: 4 SASI-HS01-00112524 (purchased from Sigma)

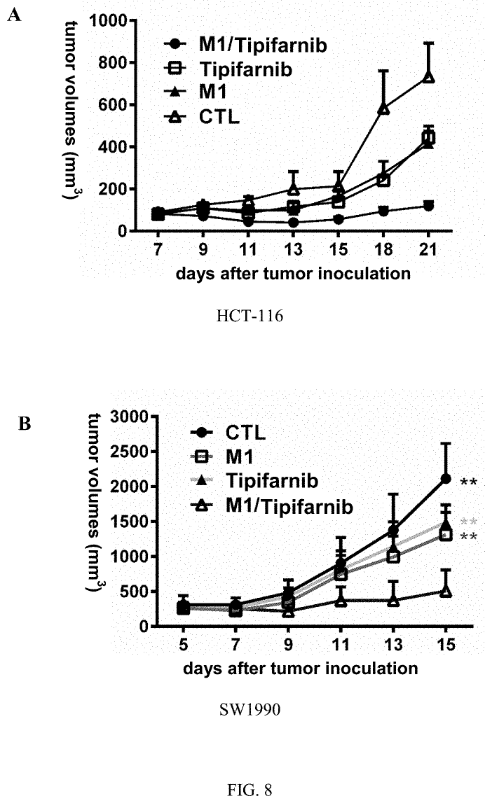

Result: the expression of structural protein E1 and nonstructural protein NS3 of the virus increased significantly after the HMGCR, the farnesyltransferase subunit FNTB and the geranylgeranyltransferase RABGGTB thereof were intefered, whereas interfering the cholesterol synthesis pathways SQLE and PGGT1B had no significant effect on the viral proteins (FIG. 7).

### EXAMPLE 8 TIPIFARNIB COMBINED WITH M1 VIRUS SIGNIFICANTLY INHIBITED THE GROWTH OF XENOGRAFTS OF HUMAN INTESTINAL AND PANCREATIC CELL CARCINOMA STRAINS

### Materials

M1 virus (Accession number CCTCC V201423), human hepatoma cell strain HCT-116 (purchased from ATCC), human pancreatic cancer cell strain SW 1990 (purchased from ATCC), and 4 week old female BALB/c nude mice.

### Method

This experiment used a randomized, single-blind design. 5×10⁶ HCT-116 or SW 1990 cells were injected subcutaneously into the dorsal flank of the 4 week old BALB/c nude mouse.

When the tumor size reached 50 mm3, the mice were divided into three groups, including untreated control group, Tipifarnib alone group (500 μg/kg/d by intraperitoneal injection), M1 infection alone group (2×10⁹ PFU/kg/d by tail vein injection of M1 virus) and Tipifarnib/M1 combined group (same dose of Tipifarnib and M1 virus), and treated with 6 consecutive injections. The length, width and weight of the tumor were measured every two days, and the volume of the tumor was measured according to the formula (length×width²)/2. One way ANOVA was performed after measuring tumor volume, ***indicates p<0.001.

### Result

As shown in FIG. 8, tumor volumes in two tumor cell transplanted tumor animals with pathological tumor dissection showed that treatment with Tipifarnib alone and M1 alone resulted in only a slight reduction in tumor volume compared to the control group, whereas treatment with Tipifarnib/M1 in combination resulted in a significant reduction in tumor volume, and one way ANOVA showed statistical differences.

The described embodiments of the present disclosure are merely illustrative examples, and the embodiments of the present disclosure are not limited to the above, and any other changes, modifications, substitutions, combinations, and simplifications that may be made without departing from the spirit and principles of the present disclosure are intended to be equivalent and fall within the scope of the present disclosure.

### REFERENCES

[1]. Ponroy, N., et al., Statins demonstrate a broad anti-cytomegalovirus activity in vitro in ganciclovir-susceptible and resistant strains. J Med Virol, 2015. 87(1): p. 141-53.

[2]. Amet, T., et al., Statin-induced inhibition of HIV-1 release from latently infected U1 cells reveals a critical role for protein prenylation in HIV-1 replication. Microbes Infect, 2008. 10(5): p. 471-80.

[3]. Mackenzie, J. M., A. A. Khromykh and R. G. Parton, Cholesterol manipulation by West Nile virus perturbs the cellular immune response. Cell Host Microbe, 2007. 2(4): p. 229-39.

[4]. Farassati, F., A. D. Yang and P. W. Lee, Oncogenes in Ras signalling pathway dictate host-cell permissiveness to herpes simplex virus 1. Nat Cell Biol, 2001. 3(8): p. 745-50.

1-10. (canceled)

11. A method of treating a human subject with tumor, comprising administering to the subject an alphavirus and a mevalonate metabolic pathway inhibitor.

12. The method of claim 11, wherein the mevalonate metabolic pathway inhibitor comprises
a substance that inhibits a pathway initiated from acetoacetyl-CoA up to farnesyl pyrophosphate, and/or
a protein farnesyl modification pathway inhibitor, and/or
a geranylgeranylation modification pathway inhibitor.

13. The method of claim 11, wherein the mevalonate metabolic pathway inhibitor comprises
a substance that inhibits the activity or formation of any one or more of acetoacetyl-CoA, acetyl-CoA, 3-hydroxy-3-methylglutaryl-CoA, mevalonate, phosphomevalonate, pyrophosphomevalonate, isopentenylpyrophosphate, dimethylacryldiphosphate, geranylpyrophosphate, and farnesylpyrophosphate, and/or
a substance that inhibits the activity or formation of any one or more of HMG-CoA synthase, HMG-CoA reductase, mevalonate kinase, phosphomevalonate kinase, mevalonate diphosphate decarboxylase, and farnesyl diphosphate synthase.

14. The method of claim 11, wherein the mevalonate metabolic pathway inhibitor comprises
a substance that inhibits activity of HMG-CoA reductase, or a substance that degrades HMG-CoA reductase, or a genetic tool that decreases the HMG-CoA reductase level, or any combination thereof; and/or
a substance that inhibits activity of farnesyltransferase, or a substance that degrades farnesyltransferase, or a genetic tool that reduces farnesyltransferase levels; and/or
a substance that inhibits activity of geranylgeranyltransferase, or a substance that degrades geranylgeranyltransferase, or a genetic tool that reduces geranylgeranyltransferase levels, or any combination thereof.

15. The method of claim 11, wherein the mevalonate metabolic pathway inhibitor comprises an HMG-CoA reductase inhibitor.

16. The method of claim 15, wherein the HMG-CoA reductase inhibitor comprises a statin compound.

17. The method of claim 15, wherein the HMG-CoA reductase inhibitor comprises
a compound selected from at least one of pravastatin, fluvastatin, lovastatin, simvastatin, atorvastatin, cerivastatin, rosuvastatin, and pitavastatin calcium, or
a derivative thereof having an HMG-CoA reductase inhibitory effect, or
a pharmaceutically acceptable salt, solvate, tautomer, or isomer thereof

18. The method of claim 11, wherein the mevalonate metabolic pathway inhibitor comprises a farnesyltransferase inhibitor.

19. The method of claim 18, wherein
the farnesyltransferase inhibitor is selected from one or more of quinolinone, benzodiazepine, and arylpyrrole; and/or
the farnesyltransferase inhibitor is selected from
at least one of Tipifarnib, FTI277, L-70472, J-104135, A-166120, and manumycin, or
a derivative thereof having a farnesyltransferase inhibitory effect, or
a pharmaceutically acceptable salt, solvate, tautomer, or isomer; and/or
the farnesyltransferase inhibitor is selected from at least one of CVFM and CIFM.

20. The method of claim 18, wherein the farnesyltransferase inhibitor is a farnesyltransferase subunit FNTB inhibitor.

21. The method of claim 11, wherein the mevalonate metabolic pathway inhibitor comprises a nucleotide consisting of at least one of the sequence of:
SEQ ID No: 1: AACCCAAUGCCCAUGUUCCdTdT;
SEQ ID No: 2: ACGACTCGGTGGAAACAGT;
and
SEQ ID No: 3: CGAGTTCTTTCACCTACTA.

22. The method of claim 11, wherein the mevalonate metabolic pathway inhibitor comprises
at least one of Tipifarnib, FTI277, fluvastatin and atorvastatin, or
a derivative thereof having a mevalonate metabolic pathway inhibitory effect, or
a pharmaceutically acceptable salt, solvate, tautomer, or isomer thereof.

23. The method of claim 11, wherein the mevalonate metabolic pathway inhibitor comprises a geranylgeranyltransferase inhibitor.

24. The method of claim 11, wherein the mevalonate metabolic pathway inhibitor comprises
a geranylgeranyl pyrophosphate inhibitor; and/or
a geranylgeranyl diphosphate synthase 1 inhibitor; and/or
Rab geranylgeranyltransferase subunit beta inhibitor.

25. The method of claim 11, wherein the mevalonate metabolic pathway inhibitor comprises
at least one selected from a group consisting of an antibody, antibody functional fragment, peptide and peptoids; and/or
at least one selected from a group consisting of gene interference material, gene editing material, gene silencing material and gene knockout material; and/or
at least one selected from a group consisting of DNA, RNA, PNA and DNA-RNA-hybrid; and/or
at least one selected from a group consisting of siRNA, dsRNA, miRNA, shRNA and ribozyme.

26. The method of claim 11, wherein the alphavirus is selected from at least one of M1 virus and Getah virus.

27. The method of claim 11, wherein a nucleotide sequence of the alphavirus has at least 97.8% homology to a nucleotide sequence of M1 virus deposited with Accession No. CCTCC V201423.


SEQUENCE LISTING (3 sequences)

SEQ ID NO: 1 - DNA, 23 bp, Artificial Sequence
  synthetic oligonucleotide
aacccaaugcccauguuccdtdt

SEQ ID NO: 2 - DNA, 19 bp, Artificial Sequence
  synthetic oligonucleotide
acgactcggtggaaacagt

SEQ ID NO: 3 - DNA, 19 bp, Artificial Sequence
  synthetic oligonucleotide
cgagttctttcacctacta